(19) United States
(12) Patent Application Publication
(10) Pub. No.: US 2004/0066490 A1
(43) Pub. Date: Apr. 8, 2004

(54) Ophthalmic refractor lens battery

(76) Inventor: David J. Stewart, Williamsville, NY (US)

Correspondence Address: George L. Snyder, Jr., Hodgson Russ LLP, Suite 2000, One M&T Plaza, Buffalo, NY, 14203-2391, US

(21) Appl. No.: 10/263,590

(22) Filed: Oct. 3, 2002

(51) Int. Cl. A61B 3/10

(52) U.S. Cl. 351/216

(57) ABSTRACT

A cylinder axis control knob for a lens battery of a subjective ophthalmic refractor comprises at least one shaped pointer formed integrally with the knob to avoid assembly problems associated with printed pointers on the knob and provide a better indication to the operator.

### FIELD OF INVENTION

The present invention relates generally to the field of ophthalmic instruments, and more particularly to subjective ophthalmic refractors for evaluating refractive characteristics of a patient's eye.

### BACKGROUND OF THE INVENTION

Subjective ophthalmic refractors of the prior art typically comprise left-eye and right-eye lens batteries each having a defined viewing path along which an operator selectively introduce one or more testing lenses having known refractive properties. During examination, the patient is positioned in a darkened room with his or her eyes aligned to view a projected target chart along the viewing paths of the left-eye and right-eye batteries. The operator then performs well-known refracting procedures, including refraction using astigmatic charts and the Jackson cross-cylinder test. A goal of the examination procedure is to determine the sphere power, cylinder power, and cylinder axis of each eye so that suitable corrective lenses may be prescribed.

With specific regard to determining the cylinder axis, it is well known to provide a rotatable cylinder axis control knob that is mechanically connected through a gear train to rotatable lens cells which hold cylinder testing lenses selectively aligned in the viewing path. When the cylinder axis control knob is rotated, a corresponding 1:1 rotation of the cylinder lens cells occurs, thereby causing the angular orientation of the cylinder axis to change. A fixed angular scale is arranged coaxially with the rotated lens cells on the viewing path, or coaxially with the cylinder axis adjustment knob. In the former arrangement, two diametrically opposite index pointer markings are applied to an outer cylinder lens in alignment with the true cylinder axis, and the pointer markings rotate with the lens cell relative to the fixed angular scale to visually indicate the precise angular orientation of the cylinder axis. An example of this type of arrangement is found in U.S. Pat. No. 2,968,213. In the latter arrangement, two diametrically opposite index pointer markings are applied to the cylinder axis adjustment knob for rotation relative to the fixed angular scale. An example of this latter type of arrangement, as well as the former type, is found in the Ultramatic RX Masters™ PHOROPTOR® refracting instrument and the Illuminated PHOROPTOR® refracting instrument offered by Reichert Ophthalmic Instruments, a division of Leica Microsystems Inc.

Under current manufacturing procedure, pointer markings are applied to an annular flange on the cylinder axis control knob by a printing process. A problem recognized by applicant is that limitations in the precision and accuracy of the printing process cause slight variations in the position of the pointer markings relative to the control knob and to each other. Consequently, during assembly, a technician often must try several knobs to find an acceptable knob having pointer markings in correct alignment with the fixed angular scale markings and with each other. This slows assembly and adds cost to the instrument.

### BRIEF SUMMARY OF THE INVENTION

Therefore, it is an object of the present invention to provide a cylinder axis control knob that can be manufactured in production lots without giving rise to downstream assembly delays associated with difficulties in aligning the cylinder axis pointers relative to an angular cylinder axis scale on a refractor lens battery.

The present invention is embodied in a subjective ophthalmic refractor lens battery having a viewing path, means for positioning at least one cylinder lens in the viewing path, a rotatable cylinder axis control knob operatively connected to the cylinder lens or lenses for rotating same about the viewing path in response to rotation of the cylinder axis control knob, and an angular cylinder axis scale coaxial with the cylinder axis control knob. In accordance with a preferred embodiment of the present invention, the cylinder axis control knob includes two integrally formed and diametrically opposite pointers that extend in a radial direction of the knob and overlap with the cylinder axis scale. The pointers are preferably tapered in three dimensions to provide an accurate reading regardless of the angle at which the operator views the cylinder axis scale and superimposed pointers. A recess is preferably provided near each pointer in alignment with a radial direction of the pointer for receiving paint or other marking material.

### BRIEF DESCRIPTION OF THE DRAWINGS

The nature and mode of operation of the present invention will now be more fully described in the following detailed description of the invention taken with the accompanying drawing figures, in which:

FIG. 1 is a front elevational view of a subjective ophthalmic refractor formed in accordance with a preferred embodiment of the present invention;

FIG. 2 is a front elevational view of a right-eye lens battery of the ophthalmic refractor shown in FIG. 1;

FIG. 3 is a perspective view of a cylinder axis control knob of the lens battery shown in FIG. 2;

FIG. 4 is a top plan view of the cylinder axis control knob; and

FIG. 5 is a cross-sectional view of the cylinder axis control knob taken generally along the line A-A in FIG. 4.

### DETAILED DESCRIPTION OF THE INVENTION

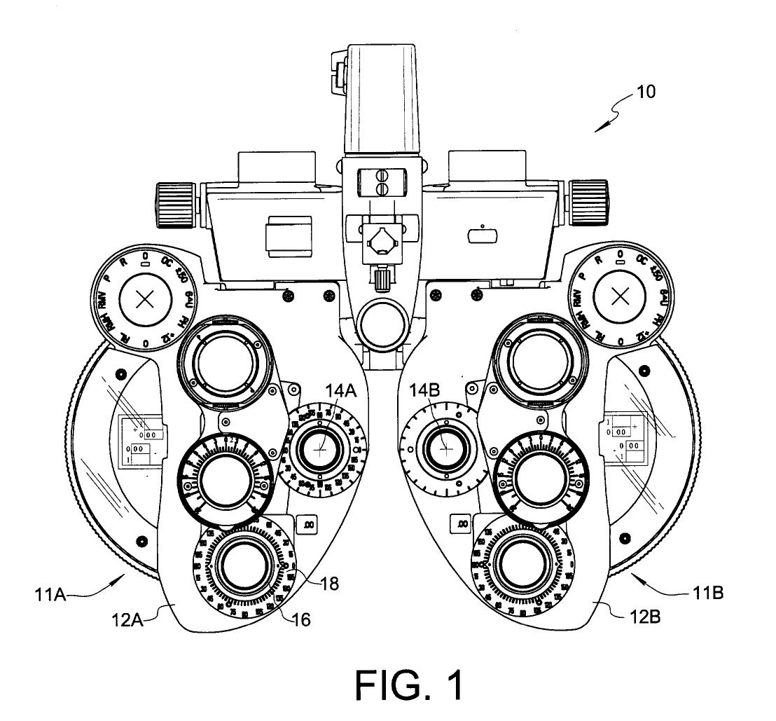

Attention is directed initially to FIG. 1 of the drawings, wherein a subjective ophthalmic refractor incorporating the present invention is shown and designated broadly by the reference numeral 10. Refractor 10 is conventional in that it generally comprises a right-eye lens battery 11A and a left-eye lens battery 11B that are mirror images of each other. Lens batteries 11A and 11B comprise respective housings 12A and 12B, as well as respective viewing paths 14A and 14B along which a patient facing a back side of refractor 10 gazes during examination. The construction and operation of ophthalmic refractor 10 are generally and substantially as taught in U.S. Pat. Nos. 2,968,213 and 2,995,065, both these patents being incorporated herein by reference.

The present invention involves the cylinder axis adjustment system found in each lens battery 11A and 11B. For sake of simplicity, the invention is described with respect to right-eye lens battery 11A only, it being understood that left-eye lens battery 11B is a mirror image of right-eye lens battery 11A.

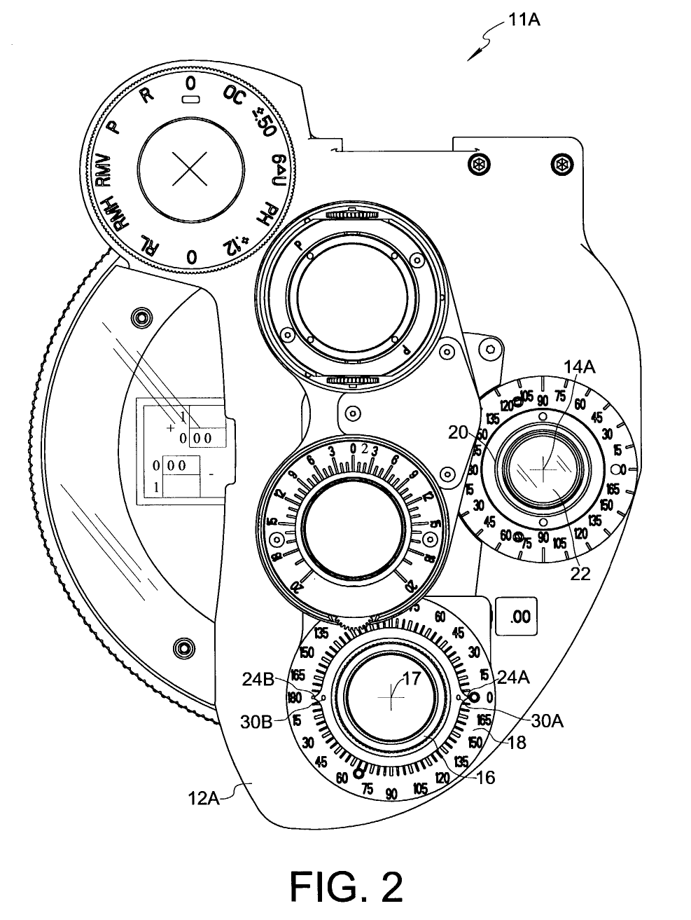

As best seen in FIG. 2, lens battery 11A includes a cylinder axis control knob 16 rotatable about a central axis 17 and surrounded by an angular cylinder axis scale 18 that is fixed relative to housing 12A and positioned to be coaxial with rotational axis 17 of cylinder axis control knob 16. Cylinder axis control knob 16 is operatively connected by way of a gear train (not shown) contained within housing 12A to lens cells 20 aligned along viewing path 14A. The lens cells 20, only one of which is visible in FIG. 2, are rotatable about viewing path 14A by way of the gear train such that rotation of cylinder axis control knob 16 causes a corresponding 1:1 rotation of lens cells 20 about viewing path 14A. Lens cells 20 each carry a cylindrical lens 22 of known power or no lens for zero power. It is well known to those skilled in the art to align a selected cylindrical lens of relatively strong power and a selected cylindrical lens of relatively weak power along viewing path 14A to achieve a desired resultant cylinder power for test purposes. The respective cylinder axes of cylindrical lenses 22 have the same angular orientation with respect to viewing path 14A, and thus the cylinder axes may be referred to as simply the cylinder axis. Thus, when cylinder axis control knob 16 is rotated, the cylinder axis associated with viewing path 14A rotates in corresponding fashion about the viewing path to change the angular orientation of the cylinder axis.

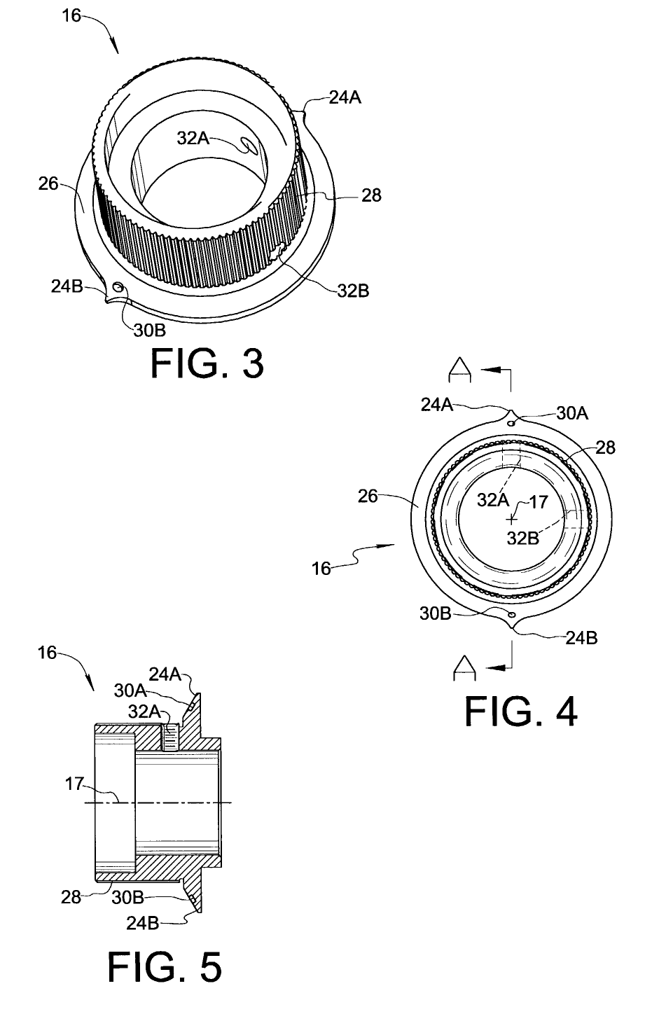

The orientation of the cylinder axis is indicated on cylinder axis scale 18. In accordance with a preferred embodiment of the present invention, and with reference to FIGS. 3-5, cylinder axis control knob 16 includes a pair of pointers 24A and 24B integrally formed therewith and arranged in diametrically opposite relation to each other about rotational axis 17. More specifically, knob 16 includes an annular flange 26 sloped toward cylinder axis scale 18 as the flange expands radially outward from a cylindrical main body 28 of knob 16, and pointers 24A and 24B project radially outward from flange 26 to overlap with cylinder axis scale 18. As will be appreciated, the slope of flange 26 is continued over the projecting pointers 24A and 24B such that the pointers can be considered to be tapered in three-dimensions rather than merely two-dimensions. This feature is important because it minimizes possible error in viewing the true location of the pointer tip on cylinder axis scale 18 due to differences in operator viewing angle.

It is preferred that pointers 24A and 24B protrude radially from cylinder axis control knob 16 sufficiently to overlap with scale line markings located at regular angular intervals about cylinder axis scale 18, as can be seen in FIG. 2. This gives the operator a better indication of the cylinder axis orientation, as compared with the prior art, especially when the pointers are between scale line markings. This feature is particularly helpful where the cylinder axis scale is bright and the scale markings are dark, such as when the cylinder axis scale is illuminated or is formed of photoluminescent material, as taught for example in commonly owned U.S. patent application Ser. No. 10/037326 filed Nov. 9, 2001, which patent is hereby incorporated by reference. As will be appreciated, a silhouette of each pointer will be readily visible against the illuminated or luminous scale.

As an additional structural feature of the preferred embodiment, cylinder axis control knob 16 includes recesses 30A and 30B formed in flange 26 and aligned along a radial pointing direction of pointers 24A and 24B, respectively. The recesses are intended to receive paint or other marking material as an added indicator.

Cylinder axis control knob 16 further includes a pair of threaded holes 32A and 32B extending radially through main body 28 for receiving set screws (not shown) for securing the knob to an actuating shaft (not shown) on which knob 16 is mounted. Threaded hole 30A is angularly aligned with pointer 24A, while threaded hole 30B is spaced 90° from threaded hole 30A. Currently, it is preferred to manufacture cylinder axis control knob 16 from aluminum by precision machining a length of cylindrical bar stock.

What is claimed is:

1. An ophthalmic refractor lens battery comprising:
a viewing path;
means for positioning at least one cylinder lens in said viewing path;
a rotatable cylinder axis control knob operatively connected to said at least one cylinder lens for rotating said at least one cylinder lens about said viewing path in response to rotation of said cylinder axis control knob; and
an angular cylinder axis scale coaxial with said cylinder axis control knob;
wherein said cylinder axis control knob has at least one integrally formed pointer=extending in a radial direction of said cylinder axis control knob and overlapping with said cylinder axis scale.

2. The ophthalmic refractor lens battery according to claim 1, wherein said at least one pointer comprises a pair of pointers spaced 180 degrees from one another about a rotational axis of said cylinder axis control knob.

3. The ophthalmic refractor lens battery according to claim 1, wherein said cylinder axis scale surrounds said cylinder axis control knob, and said at least one pointer points in an outward radial direction.

4. The ophthalmic refractor lens battery according to claim 1, wherein said at least one pointer is tapered in three-dimensions.

5. The ophthalmic refractor lens battery according to claim 4, wherein said cylinder axis control knob includes an annular flange adjacent to and sloped toward said cylinder axis scale, and said at least one pointer is integral with said flange.

6. The ophthalmic refractor lens battery according to claim 1, wherein said at least one pointer overlaps angularly spaced scale markings of said cylinder axis scale.

7. The ophthalmic refractor lens battery according to claim 2, wherein said cylinder axis scale is illuminated or is formed of photoluminescent material.

8. The ophthalmic refractor lens battery according to claim 1, wherein said cylinder axis control knob includes a recess in alignment with a radial direction of said pointer for receiving marking material.

9. The ophthalmic refractor lens battery according to claim 8, wherein said cylinder axis control knob includes an annular flange adjacent to and sloped toward said cylinder axis scale, and said recess is formed in said flange.

10. A combination comprising:
a cylinder axis scale having a plurality of scale markings angularly spaced about a central axis, wherein said cylinder axis scale includes a bright background and said scale markings are dark against said bright background;
a cylinder axis control knob rotatable about said axis relative to said cylinder axis scale for adjusting angular orientation of a cylinder lens in a viewing path of a subjective ophthalmic refractor, wherein said cylinder axis control knob has at least one integrally formed pointer extending in a radial direction of said cylinder axis control knob and overlapping with said cylinder axis scale;
whereby a silhouette of said at least one pointer may be seen against said bright background.

12. The combination according to claim 11, wherein said cylinder axis scale is illuminated from a backside thereof.

13. The combination according to claim 11, wherein said cylinder axis scale is formed of photoluminescent material.
